SEQUENCE LISTING (29 sequences)

SEQ ID NO: 1 - DNA, 107 bp, Potato virus X
  potexvirus potato virus X (PVX) engineered RNA translocator (ERT) 1-107
gaaaactaaaccatacaccaccaacacaaccaaacccaccacgcccaattgttacacacccgcttgaaaaagaaagtttaacaaatggccaaggtgcgcgaggttta

SEQ ID NO: 2 - DNA, 182 bp, Potato virus X
  potexvirus potato virus X (PVX) engineered RNA translocator (ERT) 1-182
gaaaactaaaccatacaccaccaacacaaccaaacccaccacgcccaattgttacacacccgcttgaaaaagaaagtttaacaaatggccaaggtgcgcgaggtttaccaatcttttacagactccaccacaaaaactctcatccaagatgaggcttatagaaacattcgccccatcatgga

SEQ ID NO: 3 - DNA, 336 bp, Potato virus X
  potexvirus potato virus X (PVX) engineered RNA translocator (ERT) 1-336
gaaaactaaaccatacaccaccaacacaaccaaacccaccacgcccaattgttacacacccgcttgaaaaagaaagtttaacaaatggccaaggtgcgcgaggtttaccaatcttttacagactccaccacaaaaactctcatccaagatgaggcttatagaaacattcgccccatcatggaaaaacacaaactagctaacccttacgctcaaacggttgaagcggctaatgatctagaggggttcggcatagccaccaatccctatagcattgaattgcatacacatgcagccgctaagaccatagagaataaacttctagaggtgcttggttcc

SEQ ID NO: 4 - DNA, 548 bp, Potato virus X
  potexvirus potato virus X (PVX) engineered RNA translocator (ERT) 1-548
gaaaactaaaccatacaccaccaacacaaccaaacccaccacgcccaattgttacacacccgcttgaaaaagaaagtttaacaaatggccaaggtgcgcgaggtttaccaatcttttacagactccaccacaaaaactctcatccaagatgaggcttatagaaacattcgccccatcatggaaaaacacaaactagctaacccttacgctcaaacggttgaagcggctaatgatctagaggggttcggcatagccaccaatccctatagcattgaattgcatacacatgcagccgctaagaccatagagaataaacttctagaggtgcttggttccatccgcggatctagtaaaggagaagaacttttcactggagttgtcccaattcttgttgaattagatggtgatgttaatgggcacaaattttctgtcagtggagagggtgaaggtgatgcaacatacggaaaacttacccttaaatttatttgcactactggaaaactacctgttccatggccaacacttgtcactactttctcttatggtgt

SEQ ID NO: 5 - DNA, 1797 bp, White clover mosaic virus
  potexvirus white clover mosaic virus (WClMV) engineered RNA translocator (ERT) 1-872
gaaaacaagacgagacgaacccaaacagaacgagccatccgcagaaaaactaaaccatcccaggttttctttgaacataaccaatccgtagtttgacaaaggctgccatggctaaagttcgtgccgctcttgacagaatcactgatccatcggttaaagctgtactcaatgaagaggcatacagccacatccgaccggttcttcgtgaatccctaaccaacaacccctacgccatcgcacccgatgctgctgatacgctagaaaagtatggaattgctactaatccattcgcagtgaaagtacattcccatggagcagttaaaagtattgaaaacaccctacttgagagagttgggtttaacttgccgaaagagccatgtaccttcctcttcctcaaaagaagtaagctgcgttacctcagacgtggacctagcaacaatgacatctttatcaatctagcgatcgaaccccgcgacctccaaagatacgaggaagacactcttgttgagagttggacacgtatcaccactaggtatgcatatattagtgacactttccacttcttcactaggaagatgttggctgaccttttctttcataatccggccttagatgtattatatgccactttagtacttccacccgaagccctccacaaacatcctagcatagaacctgacttatacactattaactataactttaatggtttccaatacatcccaggcaatcatggtggtggttcctactcccatgaattcaaacaactggagtggctcaaagttggacatctcaaatctccagaactatgcctcactttccagatgatcgaatccattggtgccaaccaccttttcatgattacccgcggtcaagtaaaggagaagaacttttcactggagttgtcccaattcttgttgaattagatggtgatgttaatgggcacaaattttctgtcagtggagagggtgaaggtgatgcaacatacggaaaacttacccttaaatttatttgcactactggaaaactacctgttccatggccaacacttgtcactactttctcttatggtgttcaatgcttttcaagatacccagatcatatgaagcggcacgacttcttcaagagcgccatgcctgagggatacgtgcaggagaggaccatcttcttcaaggacgacgggaactacaagacacgtgctgaagtcaagtttgagggagacaccctcgtcaacaggatcgagcttaagggaatcgatttcaaggaggacggaaacatcctcggccacaagttggaatacaactacaactcccacaacgtatacatcatggccgacaagcaaaagaacggcatcaaagccaacttcaagacccgccacaacatcgaagacggcggcgtgcaactcgctgatcattatcaacaaaatactccaattggcgatggccctgtccttttaccagacaaccattacctgtccacacaatctgccctttcgaaagatcccaacgaaaagagagaccacatggtccttcttgagtttgtaacagctgctgggattacacatggcatggatgaactatacaaacatcgcgacgcaaaacgccgtggcaccaacgttgtcaactctgttgaaatcactaacggtcgctccgaccctattggtccccttattacctatccccagtaattgccttatcacttcacttaatatgtgtggctttctgtttaataaaatttccgaaaaaaaaaaaaaaaaaaaaaaaaaaaaaaaaaaaaaaaaaaaaaaaaaaaaaaaaa

SEQ ID NO: 6 - protein, 226 aa, Potato virus X
  potexvirus potato virus X (PVX) triple gene block protein 1 (TGBp1)
MDILIISLKSLGYSRTHKSLDSGPLVVHAVAGAGKSTALRKLILRHPTFTVHTLGVPDKVSIRTRGIQKPGPIPEGNFAILDEYTLDNTTRNSYQALFADPYQAPEFSLEPHFYLETSFRVPRKVADLIAGCGFDFETNSQEEGHLEITGIFKGPLLGKVIAIDEESETTLSKHGVEFVKPCQVTGLEFKVVTVVSAAPIEEIGQSTAFYNAITRSKGLTYVRAGT

SEQ ID NO: 7 - DNA, 681 bp, Potato virus X
  potexvirus potato virus X (PVX) triple gene block protein 1 (TGBp1)
atggatattctcatcattagtttgaaaagtttaggttattctaggactcataaatctttagattcaggacctttggtagtacatgcagtagccggagccggtaagtccacagccctaaggaagttgatcctcagacacccaacattcactgtgcatacactcggtgtccctgacaaggtgagcatcagaactagaggcatacagaagccaggacctattcctgagggcaatttcgcaatcctcgatgagtatactttggacaacaccacaaggaactcataccaggcactttttgctgacccttatcaggcacctgagtttagcctagagccccacttctacttggaaacctcatttcgagttccgaggaaagtggcagatttgatagcaggctgtggcttcgatttcgagaccaactcacaggaagaagggcacttagagatcactggcatatttaaagggcccctacttgggaaggtgatagccattgacgaggagtccgagacaacactgtccaaacatggtgttgagtttgttaagccctgccaagtgactggacttgagttcaaagtagtcactgttgtgtctgccgcaccaatagaggaaattggccagtccacagctttctacaacgccatcaccaggtcaaagggactgacatatgtccgcgcagggacatag

SEQ ID NO: 8 - protein, 115 aa, Potato virus X
  potexvirus potato virus X (PVX) triple gene block protein 2 (TGBp2)
MSAQGHRLTAPVNSEKVYIVLGLSFALVSITFLLSRNSLPHVGDNIHSLPHGGAYRDGTKAILYNSPNLGSRVSLHNGKNAAFAAVLLLTLLIYGSKYISQRNHTCACGNNHSSH

SEQ ID NO: 9 - DNA, 348 bp, Potato virus X
  potexvirus potato virus X (PVX) triple gene block protein 2 (TGBp2)
atgtccgcgcagggacatagactcaccgctccggtcaattctgaaaaagtgtacatagtattaggtctatcatttgctttagtttcaattacctttctgctttctagaaatagtttaccccacgtaggtgataacattcacagtttaccacacggaggagcttacagagacggcaccaaagcaatcttgtacaactcaccaaatctagggtcacgagtgagtctacacaacggaaagaacgcagcatttgctgccgttttactactgactttactgatctatggaagcaaatacatatctcaacgcaatcatacttgtgcttgtggtaacaatcatagcagtcattag

SEQ ID NO: 10 - protein, 70 aa, Potato virus X
  potexvirus potato virus X (PVX) triple gene block protein 3 (TGBp3)
MEANTYLNAIILVLVVTIIAVISTSLVRTEPCVIKITGESITVLACKLDAETIRAIADLKPLSVERLSFH

SEQ ID NO: 11 - DNA, 213 bp, Potato virus X
  potexvirus potato virus X (PVX) triple gene block protein 3 (TGBp3)
atggaagcaaatacatatctcaacgcaatcatacttgtgcttgtggtaacaatcatagcagtcattagtacttccttagtgaggactgaaccttgtgtcatcaagattactggagaatcaatcacagtgttggcttgcaaattagatgcagaaaccatcagagccattgccgatctcaagccactctccgttgaacggttaagtttccattga

SEQ ID NO: 12 - protein, 237 aa, Potato virus X
  potexvirus potato virus X (PVX) coat protein (CP)
MSAPASTTQATGSTTSTTTKTAGATPATASGLFTIPDGDFFSTARAVVASDAVATNEDLSEIEAVWKDMKVPTDTMAQAAWDLVRHCADVGSSAQTEMIDTGPYSNGISRARLAAAIKEVCTLRQFCMKYAPVVWNWMLTNNSPPANWQAQGFKPEHKFAAFDFFNGVTNPAAIMPKEGLIRPPSEAEMNAAQTAAFVKITKARAQSNDFASLDAAVTRGRITGTTTAEAVVTLPPP

SEQ ID NO: 13 - DNA, 714 bp, Potato virus X
  potexvirus potato virus X (PVX) coat protein (CP)
atgtcagcaccagctagcacaacacaggccacagggtcaactacctcaactaccacaaaaactgcaggcgcaactcctgccacagcttcaggactgttcaccatcccggatggggatttctttagtacagcccgtgctgtagtagccagcgatgccgttgcgacgaatgaggacctcagcgagattgaggctgtctggaaggacatgaaggtgcccacagacactatggcacaggctgcttgggacttagtcagacactgtgctgatgtgggctcatctgctcaaacagaaatgatagatacgggtccctactccaacggcatcagcagagccagactggcagcagcaatcaaagaggtgtgcacacttaggcaattttgcatgaagtatgccccagtggtatggaactggatgctgactaacaacagtccacctgctaactggcaagcgcaaggtttcaagcctgagcacaaattcgctgcattcgacttcttcaatggagtcaccaacccagctgccatcatgcccaaagaggggctcattcggccaccgtctgaagctgaaatgaatgctgcccaaactgctgcctttgtgaagattacaaaggccagggcacaatccaacgactttgccagcctagatgcagctgtcactcgaggtcgtatcactggaacaacaaccgctgaggctgttgtcactctaccaccaccataa

SEQ ID NO: 14 - protein, 236 aa, White clover mosaic virus
  potexvirus white clover mosaic virus (WClMV) triple gene block protein 1 (TGBp1)
MDHIHHLLSSHGFTRTRLAKSKPIVVHAIAGSGKSTVIRKILSDLPNARAYTLGKPDPYSLSNPTIKAFAQFKRGTLDILDEYGQLPFADLDSSFEFIFTDPYQAPTDNLFEPHYTLEITYRFGPNTCNLLNQAFQSNITSLVTQDNISFGSPYLVDPVGTILAFQPDTYLILCLHQAPFFKVSEVIGYQWPTVTLYLACKISEIPEEERHLLFIGLTRHTESLLILGPDAFDSSP

SEQ ID NO: 15 - DNA, 711 bp, White clover mosaic virus
  potexvirus white clover mosaic virus (WClMV) triple gene block protein 1 (TGBp1)
atggatcacattcaccacctcctcagctcccacggttttacccgcaccagactcgccaaatccaaacctatcgttgtccatgctattgcaggctccggtaaatctactgtgatcaggaaaattctctcagacctacccaacgctagagcctacacattaggtaaaccagacccctattctctctccaaccccacaattaaagctttcgcccaattcaaaagaggtacactcgacattttggatgagtacggccaactcccgttcgccgacttagattcatctttcgagtttatcttcactgacccttaccaagcaccaactgacaatctctttgaacctcactacacactagaaatcacctacagatttggccctaacacttgtaaccttctcaatcaagcattccaatctaacatcacaagccttgtcacccaggacaacatttcatttggttcaccctacttagttgacccagtaggtaccatcctcgcgtttcaacctgacacctaccttatcctttgcttacatcaagcccctttcttcaaagtttcagaagtgattggttatcagtggcctactgtaacgttgtacctagcctgcaaaatttctgagattcctgaagaagaacgtcacctcctcttcattggcctgactagacacacagaatccctccttattttaggtcctgatgcctttgattcctccccctaa

SEQ ID NO: 16 - protein, 117 aa, White clover mosaic virus
  potexvirus white clover mosaic virus (WClMV) triple gene block protein 2 (TGBp2)
MPLIPPPNPQKTYQIAVLALGLVLLLAFVLISDHSPKVGDHLHNLPFGGEYKDGTKTIKYFQRPNQHSLSKTLAKSHNTTIFLIILGLIGTLHGLHYFSNNRRISSSLHCVLCQNKH

SEQ ID NO: 17 - DNA, 354 bp, White clover mosaic virus
  potexvirus white clover mosaic virus (WClMV) triple gene block protein 2 (TGBp2)
atgcctttgattcctccccctaatccccagaaaacttaccaaattgccgtacttgctttaggattagtgctacttcttgcctttgttctaatttctgatcactctcccaaagtaggtgatcatctgcacaatcttccttttggaggtgaatacaaagacggtactaaaactatcaagtatttccaaagacccaaccaacattccctttccaaaactcttgccaaatctcacaacaccaccattttcctgatcatcttaggtttaattggtaccttgcatggacttcactactttagtaataataggcgtatatcttctagtcttcattgtgtactttgccaaaataaacactag

SEQ ID NO: 18 - protein, 66 aa, White clover mosaic virus
  potexvirus white clover mosaic virus (WClMV) triple gene block protein 3 (TGBp3)
MDFTTLVIIGVYLLVFIVYFAKINTSMCTISISGASVEISGCDNPALFEILPNLKPFDHGLSVPSI

SEQ ID NO: 19 - DNA, 201 bp, White clover mosaic virus
  potexvirus white clover mosaic virus (WClMV) triple gene block protein 3 (TGBp3)
atggacttcactactttagtaataataggcgtatatcttctagtcttcattgtgtactttgccaaaataaacactagcatgtgtactattagtatatcaggagcttctgttgaaatctcaggttgcgacaacccggctctcttcgaaatcctcccaaatctcaaaccctttgaccacgggttaagtgtgccatctatttga

SEQ ID NO: 20 - protein, 208 aa, White clover mosaic virus
  potexvirus white clover mosaic virus (WClMV) coat protein (CP)
MATTTATTPPSLTDIRALKYTSSTVSVASPAEIEAITKTWAETFKIPNDVLPLACWDLARAFADVGASSKSELTGDSAALAGVSRKQLAQAIKIHCTIRQFCMYFANVVWNIMLDTKTPPASWSKLGYKEESKFAGFDFFDGVNHPAALMPADGLIRGPSEAELLAHQTAKQVALHRDAKRRGTNVVNSVEITNGRSDPIGPLITYPQ

SEQ ID NO: 21 - DNA, 627 bp, White clover mosaic virus
  potexvirus white clover mosaic virus (WClMV) coat protein (CP)
atggcaaccaccacagcaaccactcctccatccttgacagacatccgagccctaaaatacacttcctccaccgtctcagtcgcctcacctgctgaaattgaagctatcactaaaacctgggcagaaacattcaaaattccaaatgacgtcttgcctctcgcttgttgggatctggctcgtgctttcgctgatgttggcgcttcttctaagtctgaacttactggtgactctgctgctcttgcgggtgtttcacggaaacaactggctcaagctatcaaaatccattgcaccattcgccagttctgcatgtacttcgccaatgttgtgtggaacatcatgttagataccaaaacaccgccagcatcctggtctaaactcggctataaagaagagagcaaattcgctggctttgacttctttgatggtgtcaatcatcctgctgcactcatgcctgcagacggcctcatccgtggtccttccgaagctgaactcttagcccatcaaaccgcaaaacaagtagccctccatcgcgacgcaaaacgccgtggcaccaacgttgtcaactctgttgaaatcactaacggtcgctccgaccctattggtccccttattacctatccccagtaa

SEQ ID NO: 22 - RNA, 75 nt, Artificial Sequence
  Description of Artificial Sequenceexemplary ERT sequence corresponding to PVX transcribed nucleotides 33-107
uuuggcacguggcgguaaacaaugugacccggaagaaauucaaacauauuguuaaccccaaccaccgcgaccaaa

SEQ ID NO: 23 - RNA, 75 nt, Artificial Sequence
  Description of Artificial Sequenceexemplary ERT sequence corresponding to PVX transcribed nucleotides 33-107
uuugccaccucgccgaauacaaacacacccggaugaaaaucaaaguuuuuguaaucgccaaggagcgcgagcaaa

SEQ ID NO: 24 - RNA, 77 nt, Artificial Sequence
  Description of Artificial Sequencecis-acting element for cognate virus-ERT recognition, conserved element of RNA secondary structure within PVX nucleotides 1-107
caaacccaccacgcccaauuguuacacacccgcuugaaaaagaaaguuuaacaaauggccaaggugcgcgagguuua

SEQ ID NO: 25 - RNA, 74 nt, Potato virus X
  transcribed sequence of nucleotides 33-107 of PVX genome
aaacccaccacgcccaauuguuacacacccgcuugaaaaagaaaguuuaacaaauggccaaggugcgcgagguu

SEQ ID NO: 26 - DNA, 35 bp, Artificial Sequence
  Description of Artificial SequencePVXERTGFP specific RT-PCR primer
atctagcaggatcccagtaaaggagaagaactttt

SEQ ID NO: 27 - DNA, 25 bp, Artificial Sequence
  Description of Artificial SequencePVXERTGFP specific RT-PCR primer
taggcgtcggttatgtagacgtagt

SEQ ID NO: 28 - DNA, 20 bp, Artificial Sequence
  Description of Artificial SequenceRbcS internal control RT-PCR primer
aattgctcctggctcaaatc

SEQ ID NO: 29 - DNA, 20 bp, Artificial Sequence
  Description of Artificial SequenceRbcS internal control RT-PCR primer
gcttcctcagttctttcctc

(19) United States
(12) Patent Application Publication
(10) Pub. No.: US 2005/0055737 A1
(43) Pub. Date: Mar. 10, 2005

(54) Engineered rna translocators

(76) Inventors: Tony Lough, Auckland (NZ); Richard L.S. Forster, Patumahoe (NZ); William J. Lucas, Davis, CA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(21) Appl. No.: 10/487,002

(22) PCT Filed: Aug. 12, 2002
(86) PCT No.: PCT/US02/25692

Related U.S. Application Data
(60) Provisional application No. 60/312,156, filed on Aug. 13, 2001.

(51) Int. Cl. A01H 1/00; C12N 15/82; C07H 21/02

(52) U.S. Cl. 800/278; 536/23.1; 435/468; 800/280

(57) ABSTRACT

The present invention provides methods and compositions useful for transporting nucleic acids between cells in plants.

### FIELD OF THE INVENTION

This invention relates to methods and compositions useful for transporting nucleic acids between cells in plants.

### BACKGROUND OF THE INVENTION

Asymmetric distribution of mRNA within cells is controlled by protein-RNA interaction. These nucleotide-specific cis-acting elements, or “zip codes” (Bassell, et al., FASEB J. 13:447-454 (1999)), potentiate subcellular delivery and localized protein synthesis (Jansen, R. P. FASEB J. 13, 455-466 (1999)). This process underlies a wide range of cellular and developmental events (Bassell et al, supra, Choi, S. B. et al. Nature 407:765-767 (2000); Roegiers, et al., Trends Cell Biol 10:220-224 (2000)). A new twist to RNA function is the emerging paradigm of its involvement in non-cell-autonomous control of aberrant RNA and virus/transposon challenge (Mourrain, P. et al. Cell 101:533-542 (2000); Dalmay, et al., Cell 101:543-553 (2000); Tabara, H. et al., Cell 99:123-32 (1999); Bosher, et al., Nature Cell Biol. 2:E31-6 (2000)). Here, mobile nucleotide-specific molecules mediate targeted RNA degradation, via RNA interference (RNAi) in animals (Fire, A. et al., Nature 391:806-811 (1998); Fire, A., Trends Genet. 15:358-363 (1999)) or post transcriptional gene silencing (PTGS) in plants (Napoli, et al., Plant Cell 2:279-289 (1990); Fagard, et al., Annu. Rev. Plant Physiol. Plant Mol. Biol. 51:167-194 (2000)). The plant vascular system, and specifically the phloem, serves as the conduit for this selective movement of nucleic acids to distant organs (Palauqui, et al. EMBO J. 16:4738-4745 (1997); Ruiz-Medrano, et al. Development 126:4405-4419 (1999); Xoconostle-Cázares, et al. Science 283:94-98 (1999)). Analysis of plant virus movement has provided insights into the trafficking of proteins and nucleic acid complexes within the body of the plant (Fujiwara, et al. Plant Cell 5:1783-1794 (1993); Noueiry, et al. Cell 76:925-932 (1994)).

Supracellular control of plant developmental and physiological processes is mediated at the molecular level by hormones, small molecules, proteins and ribonucleoprotein complexes (RNPs). See, e.g., Lucas, et al. Curr. Opin. Cell Biol. 7:673-680 (1995). Cell-to-cell trafficking of transcription factors, such as KNOTTED-1 (Lucas, et al. Science 270:1980-1983 (1995)) and LEAFY (Sessions, Science 289:779-782 (2000)), via plasmodesmata, demonstrated that plants have evolved a unique mechanism to control cell fate within meristematic tissues. In addition, it has recently been established that mRNA synthesised in one organ is translocated, via the phloem, to distantly-located tissues and organs. This process involves the selective entry and exit of mRNA through the plasmodesmata which interconnect the specialised companion cells to the enucleate sieve elements that comprise the conduit for long-distance transport of nutrients and information molecules. Little is currently known concerning the mechanisms that underlie the selective trafficking of proteins and RNPs through plasmodesmata. The present invention addresses this and other problems.

### BRIEF SUMMARY OF THE INVENTION

This invention provides polynucleotide sequences comprising engineered translocator (ERT) sequences. In some embodiments, the invention provide expression cassettes comprising a polynucleotide linked to a heterologous nucleic acid, wherein: the polynucleotide comprises an ERT sequence at least 70% identical to a sequence selected from the group consisting of SEQ ID NO:1, SEQ ID NO:5, SEQ ID NO:22 and SEQ ID NO:23, the polynucleotide does not include a nucleotide sequence encoding an active PVX replicase, movement protein or coat protein; and introduction of the expression cassette into a plant tissue expressing TGBp1-3 and a PVX coat protein results in transport between cells of an RNA molecule comprising ERT. In some embodiments, the polynucleotide comprises an ERT sequence at least 70% identical to SEQ ID NO:1. In some embodiments, the polynucleotide comprises an ERT sequence at least 70% identical to SEQ ID NO:5. In some embodiments, the ERT sequence is SEQ ID NO:5. In some embodiments, the ERT sequence comprises a polynucleotide at least 70% identical to SEQ ID NO:1. In some embodiments, the ERT sequence is SEQ ID NO:1. In some embodiments, the ERT sequence comprises SEQ ID NO:2. In some embodiments, the ERT sequence comprises SEQ ID NO:3. In some embodiments; the ERT sequence comprises SEQ ID NO:4.

In some embodiments, the expression cassette further comprises a promoter operably linked to the polynucleotide. In some embodiments, the promoter is constitutive. In some embodiments, the promoter is inducible or tissue-specific.

The present invention also provides cells comprising: (a) an RNA molecule comprising a polynucleotide linked to a heterologous nucleic acid, wherein the polynucleotide comprises an ERT sequence at least 70% identical to a sequence selected from the group consisting of SEQ ID NO:1, SEQ ID NO:5, SEQ ID NO:22 and SEQ ID NO:23; (b) PVX movement proteins TGBp1-3; and (c) a PVX coat protein. In some embodiments, the polynucleotide comprises an ERT sequence at least 70% identical to SEQ ID NO:1. In some embodiments, the polynucleotide comprises an ERT sequence at least 70% identical to SEQ ID NO:5. In some embodiments, the cell is a plant cell. In some embodiments, the plant cell is part of a plant.

In some embodiments, the ERT sequence comprises SEQ ID NO:1. In some embodiments, the ERT sequence comprises SEQ ID NO:2. In some embodiments, the ERT sequence comprises SEQ ID NO:3. In some embodiments, the ERT sequence comprises SEQ ID NO:4.

The invention also provides methods of mobilizing RNA molecules between cells in a plant. In some embodiments, the methods comprise expressing an RNA molecule in a plant cell, the RNA molecule comprising an ERT sequence at least 70% identical to a sequence selected from the group consisting of SEQ ID NO:1, SEQ ID NO:5, SEQ ID NO:22 and SEQ ID NO:23, wherein the ERT sequence is linked to a heterologous polynucleotide. In some embodiments, the methods further comprise expressing PVX movement proteins TGBp1-3 and a PVX coat protein in the plant cell. In some embodiments, the plant cell expressing the RNA molecule is contained in a plant tissue that is grafted onto the plant. In some embodiments, the plant is not transgenic.

In some embodiments, the PVX movement and coat proteins are expressed from a viral vector. In some embodiments, the PVX movement and coat proteins are encoded by a polynucleotide integrated into the plant genome.

In some embodiments, the ERT sequence comprises a polynucleotide at least 70% identical to SEQ ID NO:1. In some embodiments, the ERT sequence comprises a polynucleotide at least 70% identical to SEQ ID NO:5. In some embodiments, the ERT sequence comprises SEQ ID NO:1. In some embodiments, the ERT sequence comprises SEQ ID NO:5. In some embodiments, the ERT sequence comprises SEQ ID NO:2. In some embodiments, the ERT sequence comprises SEQ ID NO:3. In some embodiments, the ERT sequence comprises SEQ ID NO:4.

In some embodiments, the TGBp1-3 and the PVX coat protein are expressed from a viral genome. In some embodiments, the TGBp 1-3 and the PVX coat protein are expressed from an integrated transgene.

The invention also provides methods of identifying a nucleic acid sequence that is transported between cells in a plant. In some embodiments, the methods comprise providing at least one polynucleotide comprising a nucleic acid sequence linked to a reporter gene; introducing the polynucleotide into a target plant cell in the plant; and determining whether the reporter gene is expressed in plant cells in the plant other than the target plant cell, thereby identifying a nucleic acid sequence that is transported between cells in a plant. In some embodiments, the reporter gene is selected from green fluorescence protein, luciferase and β-glucuronidase. In some embodiments, the nucleic acid sequence is from a plant.

The invention also provides methods of mobilizing RNA molecules between cells in a plant. In some embodiments, the method comprises expressing an RNA molecule in a plant cell, the RNA molecule comprising the nucleic acid sequence identified in claim 37 linked to a heterologous polynucleotide.

### DEFINITIONS

The term “ERT sequence” refers to a polynucleotide sequence, which when expressed in the presence of movement proteins in a plant cell, is transported to adjacent cells. ERT sequences generally have fewer than about 1000 nucleotides, and preferably have fewer than 50, 300, 200 or 100 nucleotides.

The term “movement protein” refers to a protein, which when expressed in a cell, allows for transport of nucleic acids into adjacent cells.

The phrase “nucleic acid” refers to a single or double-stranded polymer of deoxyribonucleotide or ribonucleotide bases read from the 5′ to the 3′ end. Nucleic acids may also include modified nucleotides that permit correct read through by a polymerase and do not alter expression of a polypeptide encoded by that nucleic acid. Nucleic acids include sequences that do not encode a polypeptide.

The phrase “polynucleotide sequence” or “nucleic acid sequence” includes both the sense and antisense strands of a nucleic acid as either individual single strands or in the duplex. It includes, but is not limited to, self-replicating plasmids, chromosomal sequences, and infectious polymers of DNA or RNA.

The phrase “nucleic acid sequence encoding” refers to a nucleic acid which directs the expression of a specific protein or peptide. The nucleic acid sequences include both the DNA strand sequence that is transcribed into RNA and the RNA sequence that is translated into protein. The nucleic acid sequences include both the full length nucleic acid sequences as well as non-full length sequences derived from the full length sequences. It should be further understood that the sequence includes the degenerate codons of the native sequence or sequences which may be introduced to provide codon preference in a specific host cell.

The term “promoter” refers to a region or sequence determinants located upstream or downstream from the start of transcription and which are involved in recognition and binding of RNA polymerase and other proteins to initiate transcription. A “plant promoter” is a promoter capable of initiating transcription in plant cells. Such promoters need not be of plant origin, for example, promoters derived from plant viruses, such as the CaMV35S promoter, can be used in the present invention.

The term “plant” includes whole plants, shoot vegetative organs/structures (e.g. leaves, stems and tubers), roots, flowers and floral organs/structures (e.g. bracts, sepals, petals, stamens, carpels, anthers and ovules), seed (including embryo, endosperm, and seed coat) and fruit (the mature ovary), plant tissue (e.g. vascular tissue, ground tissue, and the like) and cells (e.g. guard cells, egg cells, trichomes and the like), and progeny of same. The class of plants that can be used in the method of the invention is generally as broad as the class of higher and lower plants amenable to transformation techniques, including angiosperms (monocotyledonous and dicotyledonous plants), gymnosperms, ferns, and multicellular algae. It includes plants of a variety of ploidy levels, including aneuploid, polyploid, diploid, haploid and hemizygous.

A polynucleotide sequence is “heterologous to” an organism or a second polynucleotide sequence if it originates from a foreign species, or, if from the same species, is modified from its original form. For example, a promoter operably linked to a heterologous coding sequence refers to a coding sequence from a species different from that from which the promoter was derived, or, if from the same species, a coding sequence which is different from any naturally occurring allelic variants.

A polynucleotide “exogenous to” an individual plant is a polynucleotide which is introduced into the plant by any means other than by a sexual cross. Examples of means by which this can be accomplished are described below, and include Agrobacterium-mediated transformation, biolistic methods, electroporation, in planta techniques, and the like. Such a plant containing the exogenous nucleic acid is referred to here as an T₀ generation transgenic plant. Transgenic plants that arise from sexual cross or by selfing are descendants of such a plant.

In the case of both expression of transgenes and inhibition of endogenous genes (e.g., by antisense, or sense suppression), one of skill will recognize that the inserted polynucleotide sequence need not be identical and may be “substantially identical” to a sequence of the gene from which it was derived. As explained below, these variants are specifically covered by this term.

In the case where the inserted polynucleotide sequence is transcribed and translated to produce a functional polypeptide, one of skill will recognize that because of codon degeneracy a number of polynucleotide sequences will encode the same polypeptide. In the case of polynucleotides used to inhibit expression of an endogenous gene, the introduced sequence need not be perfectly identical to a sequence of the target endogenous gene. The introduced polynucleotide sequence will typically be at least substantially identical (as determined below) to the target endogenous sequence.

Two nucleic acid sequences or polypeptides are said to be “identical” if the sequence of nucleotides or amino acid residues, respectively, in the two sequences is the same when aligned for maximum correspondence as described below. The term “complementary to” is used herein to mean that the sequence is complementary to all or a portion of a reference polynucleotide sequence.

Optimal alignment of sequences for comparison may be conducted by the local homology algorithm of Smith and Waterman Add. APL. Math. 2:482 (1981), by the homology alignment algorithm of Needleman and Wunsch J. Mol. Biol. 48:443 (1970), by the search for similarity method of Pearson and Lipman Proc. Natl. Acad. Sci. (U.S.) 85: 2444 (1988), by computerized implementations of these algorithms (GAP, BESTFIT, BLAST, FASTA, and TFASTA in the Wisconsin Genetics Software Package, Genetics Computer Group (GCG), 575 Science Dr., Madison, Wis.), or by inspection.

“Percentage of sequence identity” is determined by comparing two optimally aligned sequences over a comparison window, wherein the portion of the polynucleotide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity.

The term “substantial identity” of polynucleotide sequences means that a polynucleotide comprises a sequence that has at least 25% sequence identity. Alternatively, percent identity can be any integer from 25% to 100%. More preferred embodiments include at least: 25%, 30%, 35%, 40%, 45%, 50%, 55%, 60%, 65%, 70%, 75%, 80%, 85%, 90%, 95%, or 99%. compared to a reference sequence using the programs described herein; preferably BLAST using standard parameters, as described below. Accordingly, ERT sequences of the invention include nucleic acid sequences that have substantial identity to SEQ D NO:1, SEQ ID NO:2, SEQ ID NO:3, SEQ ID NO:4 or SEQ ID NO:5.

One of skill will recognize that values can be appropriately adjusted to determine corresponding identity of proteins encoded by two nucleotide sequences by taking into account codon degeneracy, amino acid similarity, reading frame positioning and the like. Substantial identity of amino acid sequences for these purposes normally means sequence identity of at least 40%. Preferred percent identity of polypeptides can be any integer from 40% to 100%. More preferred embodiments include at least 60%, 65%, 70%, 75%, 80%, 85%, 90%, 95%, or 99%. Most preferred embodiments include 67%, 68%, 69%, 70%, 71%, 72%, 73%, 74% and 75%. Polypeptides which are “substantially similar” share sequences as noted above except that residue positions which are not identical may differ by conservative amino acid changes. Conservative amino acid substitutions refer to the interchangeability of residues having similar side chains. For example, a group of amino acids having aliphatic side chains is glycine, alanine, valine, leucine, and isoleucine; a group of amino acids having aliphatic-hydroxyl side chains is serine and threonine; a group of amino acids having amide-containing side chains is asparagine and glutamine; a group of amino acids having aromatic side chains is phenylalanine, tyrosine, and tryptophan; a group of amino acids having basic side chains is lysine, arginine, and histidine; and a group of amino acids having sulfur-containing side chains is cysteine and methionine. Preferred conservative amino acids substitution groups are: valine-leucine-isoleucine, phenylalanine-tyrosine, lysine-arginine, alanine-valine, aspartic acid-glutamic acid, and asparagine-glutamine.

Another indication that nucleotide sequences are substantially identical is if two molecules hybridize to each other, or a third nucleic acid, under stringent conditions. Stringent conditions are sequence dependent and will be different in different circumstances. Generally, stringent conditions are selected to be about 5° C. lower than the thermal melting point (Tm) for the specific sequence at a defined ionic strength and pH. The Tm is the temperature (under defined ionic strength and pH) at which 50% of the target sequence hybridizes to a perfectly matched probe. Typically, stringent conditions will be those in which the monovalent cation concentration is about 0.033 molar at pH 7 and the temperature is at least about 60° C.

In the present invention, mRNA containing the ERT sequences of the invention can be identified in Northern blots under stringent conditions using cDNAs of the invention or fragments of at least about 100 nucleotides. For the purposes of this disclosure, stringent conditions for such RNA-DNA hybridizations are those which include at least one wash in 0.2×SSC at 63° C. for 20 minutes, or equivalent conditions. Genomic DNA or cDNA comprising genes of the invention can be identified using the same cDNAs (or fragments of at least about 100 nucleotides) under stringent conditions, which for purposes of this disclosure, include at least one wash (usually 2) in 0.2×SSC at a temperature of at least about 50° C., usually about 55° C., for 20 minutes, or equivalent conditions.

Yet another indication that nucleotide sequences are substantially identical is if two sequences form identical or similar secondary structures. Those of skill in the art will recognize that that different nucleic acid sequences can fold into an identical or a structurally equivalent secondary structure. Indeed, the primary nucleotide sequence can be altered without changing the secondary structure of the sequence.

### BRIEF DESCRIPTION OF THE DRAWINGS

FIG. 1 illustrates viral constructs based on the potexvirus genome in which GFP was inserted and used as a reporter for cell-to-cell transport of RNA.

FIG. 2 illustrates localization of the minimal cis-acting sequence essential for cell-to-cell trafficking of heterologous RNA. FIG. 2a illustrates ERT constructs developed using 540, 336, 182, 143, 107, 88 or 49 5′ viral nucleotides. FIG. 2b illustrates a conserved element of RNA secondary structure located within nucleotides 1-107 that functions as a cis-acting element essential for cognate virus-ERT recognition.

### DETAILED DESCRIPTION

1. Introduction

The present invention provides methods and compositions useful for transporting nucleic acids between cells in-plants. Engineered RNA translocator (ERT) sequences are provided which can be linked to a gene of interest. Transcripts comprising an ERT sequence are transported from one cell to other cells in a plant. Cell to cell transport is mediated by transport (“movement”) proteins. Thus, by controlling when ERT sequences or movement proteins are in a cell, the invention provides methods of controlling when and where ERT sequences are transported in a plant. Control of transport of the ERT sequences, allows for regulation of genes linked to the ERT sequence. This control also allows for transport of the sequences into cells that do not comprise transgenes inserted in their nuclei.

The invention provides for two components that can be used in combination to control transcript cell-to-cell movement. First, the invention provides ERT sequences that are recognized by movement proteins and can be moved between cells. Second, the invention provides movement proteins that act as the molecular machinery that transports the ERT sequences between cells.

2. RNA Translocator (ERT) Sequences

ERT sequences are sequences that are recognized by proteins that can transport the ERT sequence, and any linked sequence, between plant cells. ERT sequences can be derived from endogenous plant gene sequences or they can be derived from viruses. Examples of ERT sequences include sequences derived from about the first 500 base pairs, and more preferably about the first 300 or about 100 base pairs, of the genome of potato virus X (potex) viruses. Potex viruses are well known in the art and are recognized by their distinct capsid and genome structure. See, e.g., Matthews, Plant Virology 3d ed. (Academic Press, 1991); Mandahar, Molecular Biology of Plant Viruses (Kluwer Academic Press, 1999). Examples of potexviruses include, e.g., asparagus virus 3 (AV-3), cactus virus X (CVX), cassaya virus X (CsVX), clover yellow mosaic virus (ClYMV), Commelina virus X (ComVX), Cymbidium mosaic virus (CymMV), foxtail mosaic virus (FoMV), hydrangea ringspot virus (HRSV), lily virus X (LVX), Narcissus mosaic virus (NMV), Nerine virus X (NVX), papaya mosaic virus (PapMV), pepino mosaic virus (PepMV), Plantago severe mottle virus (PlSMV), plantain virus X (PlVX), potato aucuba mosaic virus (PAMV) potato virus X (PVX), tulip virus X (TVX), viola mottle virus (VMV), white clover mosaic virus (WC1MV).

Particularly preferred ERT sequences can be obtained from the potex viruses potato virus X (PVX) and white clover mosaic virus (WC1MV). For example, in some embodiments, ERT sequences comprising about the first 336 base pairs of the PVX genome, and more preferably about the first 182, or about the first 107 base pairs of the PVX genome are provided. Similarly, sequences from about the first 350 base pairs of the WC1MV genome, and more preferably about the first 150 nucleotides of the WC1MV genome are provided.

In some embodiments, the ERT sequences are active when they are transcribed into ribonucleic acids. Without intending to limit the invention to a particular theory of operation, it is believed that nucleotide sequences from position 33-107 of the PVX genome form a secondary structure recognized by the appropriate movement proteins and thus is transported between cells. Therefore, the ERT sequences of the invention include those sequences that form an equivalent secondary structure to the ERT sequences exemplified herein. For example, the transcribed sequence of nucleotides 33-107 of the PVX genome is AAACCCACCACGCCCAAUUGUUACACACCCGCUUGAAA AAGAAAGUUUAACAAAUGGCCAAGGUGCGCGAGGUU. Thus, sequences that form the same structure, such as sequences where all nucleotide binding pairs forming secondary structure are replaced with their complement, are encompassed in the ERT sequences of the invention. Two exemplary sequence corresponding to the above-listed sequence are:

UUUGGCACGUGGCGGUAAACAAUGUGACCCGGAAGAAAUUCAAACAUAUU
GUUAACCCCAACCACCGCGACCAAA
and
UUUGCCACCUCGCCGAAUACAAACACACCCGGAUGAAAAUCAAAGUUUUU
GUAAUCGCCAAGGAGCGCGAGCAAA.

Moreover, variants, derivatives and fragments of the sequences described herein can also be used as ERT sequences. Furthermore, nucleotide sequences with similar or identical secondary structure as the ERT sequences described herein can also be used as ERT sequences. For example, the secondary structure of the PVX genome has been described previously. See, e.g., Miller et al., J. Mol. Biol. 284:591-608 (1998). Since it is likely that the viral ERT and movement machinery has been modified or derived from an endogenous plant system with similar characteristics, gene that are known to move in plants will contain a sequence similar in function to the viral ERT described herein. Thus, by selecting a group of plant genes known or likely to move in plants and examining them for RNA structures substantially similar to the viral ERT sequences described herein, it is possible to identify an endogenous ERT sequence.

Endogenous plant ERT sequences can be isolated by identifying plant sequences with sequence identity or similar or identical secondary structures as the sequences disclosed herein. Secondary structure of RNA sequences can be determined as described in, e.g., “RNA Secondary Structure Prediction” In CURRENT PROTOCOLS IN NUCLEIC ACID CHEMISTRY (Beaucage, et al., eds., 2000) at 11.2.1-11.2.10; Zuker, Curr Opin Struct Biol. 10:303-310 (2000).

Functional ERT sequences can be identified as described in the Examples. Briefly, a candidate ERT sequence can be linked to a reporter gene sequence and then transformed into a plant cell with the appropriate movement proteins. For example, for ERT sequences that are mobilized by PVX movement proteins, the plant cell is further transformed with the TGBp1-3 and PVX coat protein (CP) genes. Typically, constructs encoding TGBp1-3 and the PVX coat protein and a candidate ERT sequence linked to a reporter gene are transiently expressed in plant leaf cells using biolistics. An active ERT sequence is recognized by reporter gene activity in cells adjacent to the bombarded cells. reporter genes include, e.g., green fluorescent protein, luciferase, and β-glucuronidase (GUS).

An ERT sequence can be linked to any heterologous polynucleotide that confers a desired phenotype. Preferably, the ERT sequence is linked to the heterologous sequence such that the ERT sequence is at the 5′ end of the resulting combined sequence. Heterologous polynucleotides include those that affect the chemical composition of the plant (e.g., lipid, starch, protein, vitamin content, etc.), architecture or habit of the plant (i.e., time to flowering, plant size, fruit size and quality, etc.), environment where the plant can be grown (e.g., salt, cold or heat tolerance, etc.), flowering phenotypes, fruiting phenotypes, yield, pest resistance and the like.

Heterologous polynucleotide sequences include those encoding genes involved in flowering time. Exemplary genes include, e.g., those listed in Levy & Dean Plant Cell 10:1973-1989. Other useful genes include those involved in plant architecture such as LATERAL SUPPRESSOR and TEOSINTE BRANCHED. See, e.g., Napoli et al. Current Topics in Develop. Biol. 44:127-169 (1999). In addition, members of the GRAS gene family such as SCARECROW (see, e.g., Pysh, et al. Plant J 18(1):111-9 (1999). Other useful genes include hormone synthesis genes, including the GAI family of genes (see, e.g., Ogawa, et al. Gene 245(1):21-9 (2000). Polynucleotides encoding transcription factors involved in control of secondary metabolites are also useful. Exemplary gene sequences include those of Myc and Myb transcription factors. See, e.g., Riechmann et al., Science 290:2105-2110 (2000).

3. Movement Proteins

Movement proteins recognize an ERT sequence, thereby allowing for transport of the polynucleotide sequence comprising the ERT sequence between plant cells. Without intending to limit the scope of the invention, it is believed that the movement proteins allow for the transport of ERT-containing polynucleotides through the plasmodesmota. Movement proteins can be endogenous to a particular plant, or movement proteins can be from exogenous sources, such as plant viruses. Typically, movement proteins are sequence specific, i.e., only a subset of ERT sequences are mobilized by a particular set of movement proteins.

In some embodiments, the movement proteins are the PVX movement proteins TGBp1-3 and the PVX coat protein. In some of these embodiments, all four of these proteins are expressed to mobilize ERT sequences between cells. The PVX proteins TGBp1-3 and the PVX coat protein are well known and are described in, e.g., Lough, et al., Mol. Plant-Microbe Interact. 11:801-14 (1998). Exemplary amino acid sequences (SEQ ID NOs: 6, 8, 10 and 12)) of proteins TGBp1-3 and the PVX coat protein, as well as their nucleotide sequences (SEQ ID NOs:7, 9, 11, and 13) are provided herein. Exemplary WC1MV TGBp1-3 and the coat protein amino acid sequences are also provided (e.g., SEQ ID NOs: 14, 16, 18 and 20, respectively), as well as the nucleotide sequences (SEQ ID NOs:15, 17, 19 and 21, respectively) encoding the proteins.

Additional movement proteins can be isolated from plant viral or plant sources as described herein. For example, following isolation of an endogenous plant ERT sequence, the plant gene products that enable movement of the ERT sequence can be identified by genetic or biochemical (e.g., binding) studies. Genetic assays useful for identifying plant movement components include, e.g., mutagenesis to knock out movement function, thereby identifying a component necessary for movement.

Movement proteins can be expressed in a plant cell in numerous ways. In some embodiments, movement proteins are expressed by infecting a plant with a native or recombinant virus that expresses the desired movement proteins. Alternatively, the plant can be stably or transiently transformed with an expression cassette that encodes the movement proteins.

4. Mobilizing an ERT-Containing Polynucleotide Between Plant Cells.

Mobilization of an ERT-containing sequence in a first plant cell to a second plant cell can be achieved by expressing the appropriate movement proteins in the first cell. Thus, transport of an RNA sequence containing an ERT sequence can be controlled by controlling the expression of one or more movement proteins or expression of the ERT-containing sequence.

In some embodiments, the movement proteins and ERT-containing sequence are expressed in a rootstock. In these embodiments, the scion (i.e., the portion ot a plant that is grafted to a rootstock) can optionally be non-transgenic. Transport of an ERT-containing RNA into the scion can be initiated by expression of movement proteins in the scion by endogenous movement proteins or expression of heterologous movement proteins, e.g., provided by viral infection (see, e.g., Chapman, et al., D.C. Plant J. 2, 549-557 (1992)) or other methods of transient or stable transformation. Similarly, ERT expression can be in the scion and the rootstock can be optionally non-transgenic.

Thus, new genetic material can be transferred to established plants, including non-annual plants such as apples, citrus, palm, maple or rubber trees and the like. The genetic material can be introduced by grafting a scion or other plant part containing an ERT sequence linked to a heterologous sequence and then mobilizing the RNA into the plant. Mobilization can occur by the function of endogenous plant movement proteins or by expressing viral movement proteins in the plant.

In some embodiments, an ERT sequence can be transported throughout a plant tissue, such as a stem or branch, by placing the tissue in a solution containing the ERT-containing sequence and expressing movement proteins in the tissue. Again, movement proteins can be expressed endogenously or by expression of heterologous movement proteins, e.g., via transgenes).

5. Isolation of Nucleic Acids of the Invention

The isolation of sequences of the invention, including ERT sequences and sequence encoding movement proteins, may be accomplished by a number of techniques. For instance, oligonucleotide probes based on the sequences disclosed here can be used to identify the desired gene in a cDNA or genomic DNA library from a desired species. To construct genomic libraries, large segments of genomic DNA are generated by random fragmentation, e.g. using restriction endonucleases, and are ligated with vector DNA to form concatemers that can be packaged into the appropriate vector. To prepare a library of cDNAs from a specific tissue, mRNA is isolated from that tissue and a cDNA library that contains the mRNA is prepared from the mRNA. Similarly, isolation of viral genomic polynucleotides is well known.

A cDNA or genomic library can constructed in a vector such that the library DNA is linked a reporter gene (e.g., GFP, luciferase, β-glucuronidase, and the like.). Clones from this library can be screened by introducing the clones into cells of plant tissues (e.g., by biolistic methods) and then examining the tissues for reporter gene expression beyond the cells into which the clones were introduced. These screens can be carried out on pools of clones and can be carried out robotically, thereby allowing for large numbers of candidate sequences to be screened for ERT activity.

The cDNA or genomic library can then be screened using a probe based upon the sequence of a cloned sequence such as the polynucleotides disclosed here. Probes may be used to hybridize with genomic DNA or cDNA sequences to isolate homologous genes in the same or different plant species.

Alternatively, the nucleic acids of interest can be amplified from nucleic acid samples using amplification techniques. For instance, polymerase chain reaction (PCR) technology to amplify the sequences of the genes directly from mRNA, from cDNA, from genomic libraries or cDNA libraries. PCR and other in vitro amplification methods may also be useful, for example, to clone nucleic acid sequences that code for proteins to be expressed, to make nucleic acids to use as probes for detecting the presence of the desired mRNA in samples, for nucleic acid sequencing, or for other purposes.

Appropriate primers and probes for identifying ERT sequences from samples containing virus (e.g., infected plant tissue) are generated from comparisons of the sequences provided herein. For a general overview of PCR see PCR Protocols: A Guide to Methods and Applications. (Innis, M, Gelfand, D., Sninsky, J. and White, T., eds.), Academic Press, San Diego (1990).

Polynucleotides may also be synthesized by well-known techniques as described in the technical literature. See, e.g., Carruthers et al., Cold Spring Harbor Symp. Quant. Biol. 47:411-418 (1982), and Adams et al., J. Am. Chem. Soc. 105:661 (1983). Double stranded DNA fragments may then be obtained either by synthesizing the complementary strand and annealing the strands together under appropriate conditions, or by adding the complementary strand using DNA polymerase with an appropriate primer sequence.

6. Preparation of Recombinant Vectors

To use isolated sequences in the above techniques, recombinant DNA vectors suitable for transformation of plant cells are prepared. Techniques for transforming a wide variety of higher plant species are well known and described in the technical and scientific literature. See, for example, Weising et al. Ann. Rev. Genet. 22:421-477 (1988). A DNA sequence coding for the desired polypeptide, for example a cDNA sequence encoding a full length protein, will preferably be combined with transcriptional and translational initiation regulatory sequences which will direct the transcription of the sequence from the gene in the intended tissues of the transformed plant. For example, an expression cassette comprising a promoter that is operably linked to a polynucleotide such that transcripts comprise an ERT sequence are provided herein. ERT sequences can be contained at any location of an RNA molecule, including the 5′ or 3′ end. For example, the coding sequence of the desired polypeptide can be altered to fold into the appropriate secondary structure without significantly altering the polypeptide encoded.

For overexpression, a plant promoter fragment may be employed which will direct expression of the gene in all tissues of a regenerated plant. Such promoters are referred to herein as “constitutive” promoters and are active under most environmental conditions and states of development or cell differentiation. Examples of constitutive promoters are provided below. Alternatively, the plant promoter may direct expression of the polynucleotide of the invention in a specific tissue (tissue-specific promoters) or may be otherwise under more precise environmental control (inducible promoters). Examples of tissue-specific promoters under developmental control include promoters that initiate transcription only in certain tissues, such as fruit, seeds, or flowers. Examples of environmental conditions that may affect transcription by inducible promoters include anaerobic conditions, elevated temperature, or the presence of light.

If proper polypeptide expression is desired, a polyadenylation region at the 3′-end of the coding region should be included. The polyadenylation region can be derived from the natural gene, from a variety of other plant genes, or from T-DNA.

The vector comprising the sequences (e.g., promoters or coding regions) from genes of the invention will typically comprise a marker gene which confers a selectable phenotype on plant cells. For example, the marker may encode biocide resistance, particularly antibiotic resistance, such as resistance to kanamycin, G418, bleomycin, hygromycin, or herbicide resistance, such as resistance to chlorosluforon or Basta.

The invention provides promoters operably linked to a polynucleotide comprising an ERT sequence and a heterologous polynucleotide. A variety of different expression constructs, such as expression cassettes and vectors suitable for transformation of plant cells can be prepared. Techniques for transforming a wide variety of higher plant species are well known and described in the technical and scientific literature. See, e.g., Weising et al. Ann. Rev. Genet. 22:421-477 (1988).

Sequences controlling eukaryotic gene expression have been extensively studied. For instance, promoter sequence elements include the TATA box consensus sequence (TATAAT), which is usually 20 to 30 base pairs upstream of the transcription start site. In most instances the TATA box is required for accurate transcription initiation. In plants, further upstream from the TATA box, at positions −80 to −100, there is typically a promoter element with a series of adenines surrounding the trinucleotide G (or T) N G. J. Messing et al., in Genetic Engineering in Plants, pp. 221-227 (Kosage, Meredith and Hollaender, eds. (1983)). A number of methods are known to those of skill in the art for identifying and characterizing promoter regions in plant genomic DNA (see, e.g., Jordano, et al., Plant Cell, 1: 855-866 (1989); Bustos, et al., Plant Cell, 1:839-854 (1989); Green, et al., EMBO J. 7, 4035-4044 (1988); Meier, et al, Plant Cell, 3, 309-316 (1991); and Zhang (1996) Plant Physiology 110:1069-1079).

Constitutive Promoters

A promoter fragment can be employed which will direct expression of an ERT nucleic acid in all transformed cells or tissues, e.g. as those of a regenerated plant. Such promoters are referred to herein as “constitutive” promoters and are active under most environmental conditions and states of development or cell differentiation. Promoters that drive expression continuously under physiological conditions are referred to as “constitutive” promoters and are active under most environmental conditions and states of development or cell differentiation. Examples of constitutive promoters include those from viruses which infect plants, such as the cauliflower mosaic virus (CaMV) 35S transcription initiation region (see, e.g., Dagless (1997) Arch. Virol. 142:183-191); the 1′- or 2′-promoter derived from T-DNA of Agrobacterium tumafaciens (see, e.g., Mengiste (1997) supra; O'Grady (1995) Plant Mol. Biol. 29:99-108); the promoter of the tobacco mosaic virus; the promoter of Figwort mosaic virus (see, e.g., Maiti (1997) Transgenic Res. 6:143-156); actin promoters, such as the Arabidopsis actin gene promoter (see, e.g., Huang (1997) Plant Mol. Biol. 1997 33:125-139); alcohol dehydrogenase (Adh) gene promoters (see, e.g., Millar (1996) Plant Mol. Biol. 31:897-904); ACT11 from Arabidopsis (Huang et al. Plant Mol. Biol. 33:125-139 (1996)), Cat3 from Arabidopsis (GenBank No. U43147, Zhong et al., Mol. Gen. Genet. 251:196-203 (1996)), the gene encoding stearoyl-acyl carrier protein desaturase from Brassica napus (Genbank No. X74782, Solocombe et al. Plant Physiol. 104:1167-1176 (1994)), GPc1 from maize (GenBank No. X15596, Martinez et al. J. Mol. Biol 208:551-565 (1989)), Gpc2 from maize (GenBank No. U45855, Manjunath et al., Plant Mol. Biol. 33:97-112 (1997)), other transcription initiation regions from various plant genes known to those of skill.

Inducible Promoters

Alternatively, a plant promoter may direct expression of the ERT nucleic acid of the invention under the influence of changing environmental conditions or developmental conditions. Examples of environmental conditions that may effect transcription by inducible promoters include anaerobic conditions, elevated temperature, drought, or the presence of light. Such promoters are referred to herein as “inducible” promoters. For example, the invention incorporates the drought-inducible promoter of maize (Busk (1997) supra); the cold, drought, and high salt inducible promoter from potato (Kirch (1997) Plant Mol. Biol. 33:897-909).

Alternatively, plant promoters which are inducible upon exposure to plant hormones, such as auxins, are used to express the nucleic acids of the invention. For example, the invention can use the auxin-response elements E1 promoter fragment (AuxREs) in the soybean (Glycine max L.) (Liu (1997) Plant Physiol. 115:397-407); the auxin-responsive Arabidopsis GST6 promoter (also responsive to salicylic acid and hydrogen peroxide) (Chen (1996) Plant J. 10: 955-966); the auxin-inducible parc promoter from tobacco (Sakai (1996) 37:906-913); a plant biotin response element (Streit (1997) Mol. Plant Microbe Interact. 10:933-937); and, the promoter responsive to the stress hormone abscisic acid (Sheen (1996) Science 274:1900-1902).

Plant promoters that are inducible upon exposure to chemicals reagents which can be applied to the plant, such as herbicides or antibiotics, are also used to express the nucleic acids of the invention. For example, the maize In2-2 promoter, activated by benzenesulfonamide herbicide safeners, can be used (De Veylder (1997) Plant Cell Physiol. 38:568-577); application of different herbicide safeners induces distinct gene expression patterns, including expression in the root, hydathodes, and the shoot apical meristem. Other inducible promoters include, e.g., a tetracycline-inducible promoter, e.g., as described with transgenic tobacco plants containing the Avena sativa L. (oat) arginine decarboxylase gene (Masgrau (1997) Plant J. 11:465-473); or, a salicylic acid-responsive element (Stange (1997) Plant J. 11:1315-1324.

Tissue-Specific Promoters

Alternatively, the plant promoter may direct expression of the polynucleotide of the invention in a specific tissue (tissue-specific promoters). Tissue specific promoters are transcriptional control elements that are only active in particular cells or tissues at specific times during plant development, such as in vegetative tissues or reproductive tissues. Examples of tissue-specific promoters under developmental control include promoters that initiate transcription only (or primarily only) in certain tissues, such as vegetative tissues, e.g., roots or leaves, or reproductive tissues, such as fruit, seeds, flowers, or any embryonic tissue.

Suitable seed-specific promoters are derived from the following genes: MAC1 from maize, Sheridan (1996) Genetics 142:1009-1020; Cat3 from maize, GenBank No. L05934, Abler (1993) Plant Mol. Biol. 22:10131-1038; vivparous-1 from Arabidopsis, Genbank No. U93215; atmycI from Arabidopsis, Urao (1996) Plant Mol. Biol. 32:571-57; Conceicao (1994) Plant 5:493-505; napA from Brassica napus, GenBank No. J02798, Josefsson (1987) JBL 26:12196-1301; the napin gene family from Brassica napus, Sjodahl (1995) Planta 197:264-271.

A tomato promoter active during fruit ripening, senescence and abscission of leaves and, to a lesser extent, of flowers can be used (Blume (1997) Plant J. 12:731-746). Other exemplary promoters include the pistol specific promoter in the potato (Solanum tuberosum L.) SK2 gene, encoding a pistil-specific basic endochitinase (Ficker (1997) Plant Mol. Biol. 35:425-431); the Blec4 gene from pea (Pisum sativum cv. Alaska), active in epidermal tissue of vegetative and floral shoot apices of transgenic alfalfa. This makes it a useful tool to target the expression of foreign genes to the epidermal layer of actively growing shoots.

A variety of promoters specifically active in vegetative tissues, such as leaves, stems, roots and tubers, can also be used to express the ERT nucleic acids of the invention. For example, promoters controlling patatin, the major storage protein of the potato tuber, can be used, see, e.g., Kim (1994) Plant Mol. Biol. 26:603-615; Martin (1997) Plant J. 11:53-62. The ORF13 promoter from Agrobacterium rhizogenes which exhibits high activity in roots can also be used (Hansen (1997) Mol. Gen. Genet. 254:337-343. Other useful vegetative tissue-specific promoters include: the tarin promoter of the gene encoding a globulin from a major taro (Colocasia esculenta L. Schott) corm protein family, tarin (Bezerra (1995) Plant Mol. Biol. 28:137-144); the curculin promoter active during taro corm development (de Castro (1992) Plant Cell 4:1549-1559) and the promoter for the tobacco root-specific gene TobRB7, whose expression is localized to root meristem and immature central cylinder regions (Yamamoto (1991) Plant Cell 3:371-382).

Leaf-specific promoters, such as the ribulose biphosphate carboxylase (RBCS) promoters can be used. For example, the tomato RBCS1, RBCS2 and RBCS3A genes are expressed in leaves and light-grown seedlings, only RBCS1 and RBCS2 are expressed in developing tomato fruits (Meier (1997) FEBS Lett. 415:91-95). A ribulose bisphosphate carboxylase promoters expressed almost exclusively in mesophyll cells in leaf blades and leaf sheaths at high levels, described by Matsuoka (1994) Plant J. 6:311-319, can be used. Another leaf-specific promoter is the light harvesting chlorophyll a/b binding protein gene promoter, see, e.g., Shiina (1997) Plant Physiol. 115:477-483; Casal (1998) Plant Physiol. 116:1533-1538. The Arabidopsis thaliana myb-related gene promoter (Atmyb5) described by Li (1996) FEBS Lett. 379:117-121, is leaf-specific. The Atmyb5 promoter is expressed in developing leaf trichomes, stipules, and epidermal cells on the margins of young rosette and cauline leaves, and in immature seeds. Atmyb5 mRNA appears between fertilization and the 16 cell stage of embryo development and persists beyond the heart stage. A leaf promoter identified in maize by Busk (1997) Plant J. 11: 1285-1295, can also be used.

Another class of useful vegetative tissue-specific promoters are meristematic (root tip and shoot apex) promoters. For example, the “SHOOTMERISTEMLESS” and “SCARECROW” promoters, which are active in the developing shoot or root apical meristems, described by Di Laurenzio (1996) Cell 86:423-433; and, Long (1996) Nature 379:66-69; can be used. Another useful promoter is that which controls the expression of 3-hydroxy-3methylglutaryl coenzyme A reductase HMG2 gene, whose expression is restricted to meristematic and floral (secretory zone of the stigma, mature pollen grains, gynoecium vascular tissue, and fertilized ovules) tissues (see, e.g., Enjuto (1995) Plant Cell. 7:517-527). Also useful are knI-related genes from maize and other species which show meristem-specific expression, see, e.g., Granger (1996) Plant Mol. Biol. 31:373-378; Kerstetter (1994) Plant Cell 6:1877-1887; Hake (1995) Philos. Trans. R. Soc. Lond. B. Biol. Sci. 350:45-51. For example, the Arabidopsis thaliana KNAT1 promoter. In the shoot apex, KNAT1 transcript is localized primarily to the shoot apical meristem; the expression of KNAT1 in the shoot meristem decreases during the floral transition and is restricted to the cortex of the inflorescence stem (see, e.g., Lincoln (1994) Plant Cell 6:1859-1876).

One of skill will recognize that a tissue-specific promoter may drive expression of operably linked sequences in tissues other than the target tissue. Thus, as used herein a tissue-specific promoter is one that drives expression preferentially in the target tissue, but may also lead to some expression in other tissues as well.

In another embodiment, a polynucleotide comprising an ERT nucleic acid is expressed through a transposable element. This allows for constitutive, yet periodic and infrequent expression of the nucleic acid. The invention also provides for use of tissue-specific promoters derived from viruses which can include, e.g., the tobamovirus subgenomic promoter (Kumagai (1995) Proc. Natl. Acad. Sci. USA 92:1679-1683; the rice tungro bacilliform virus (RTBV), which replicates only in phloem cells in infected rice plants, with its promoter which drives strong phloem-specific reporter gene expression; the cassaya vein mosaic virus (CVMV) promoter, with highest activity in vascular elements, in leaf mesophyll cells, and in root tips (Verdaguer (1996) Plant Mol. Biol. 31:1129-1139).

7. Production of Transgenic Plants

DNA constructs of the invention may be introduced into the genome of the desired plant host by a variety of conventional techniques. For example, the DNA construct may be introduced directly into the genomic DNA of the plant cell using techniques such as electroporation and microinjection of plant cell protoplasts, or the DNA constructs can be introduced directly to plant tissue using ballistic methods, such as DNA particle bombardment. Alternatively, the DNA constructs may be combined with suitable T-DNA flanking regions and introduced into a conventional Agrobacterium tumefaciens host vector. The virulence functions of the Agrobacterium tumefaciens host will direct the insertion of the construct and adjacent marker into the plant cell DNA when the cell is infected by the bacteria.

Microinjection techniques are known in the art and well described in the scientific and patent literature. The introduction of DNA constructs using polyethylene glycol precipitation is described in Paszkowski et al. Embo J. 3:2717-2722 (1984). Electroporation techniques are described in Fromm et al. Proc. Natl. Acad. Sci. USA 82:5824 (1985). Ballistic transformation techniques are described in Klein et al. Nature 327:70-73 (1987).

Agrobacterium tumefaciens-mediated transformation techniques, including disarming and use of binary vectors, are well described in the scientific literature. See, for example Horsch et al. Science 233:496-498 (1984), and Fraley et al. Proc. Natl. Acad. Sci. USA 80:4803 (1983).

Transformed plant cells which are derived by any of the above transformation techniques can be cultured to regenerate a whole plant which possesses the transformed genotype and thus the desired phenotype. Such regeneration techniques rely on manipulation of certain phytohormones in a tissue culture growth medium, typically relying on a biocide and/or herbicide marker that has been introduced together with the desired nucleotide sequences. Plant regeneration from cultured protoplasts is described in Evans et al., Protoplasts Isolation and Culture, Handbook of Plant Cell Culture, pp. 124-176, MacMillilan Publishing Company, New York, 1983; and Binding, Regeneration of Plants, Plant Protoplasts, pp. 21-73, CRC Press, Boca Raton, 1985. Regeneration can also be obtained from plant callus, explants, organs, or parts thereof. Such regeneration techniques are described generally in Klee et al. Ann. Rev. of Plant Phys. 38:467-486 (1987).

The nucleic acids of the invention can be used to confer desired traits on essentially any plant. Thus, the invention has use over a broad range of plants, including species from the genera Asparagus, Atropa, Avena, Brassica, Citrus, Citrullus, Capsicum, Cucumis, Cucurbita, Daucus, Fragaria, Glycine, Gossypium, Helianthus, Heterocallis, Hordeum, Hyoscyamus, Lactuca, Linum, Lolium, Lycopersicon, Malus, Manihot, Majorana, Medicago, Nicotiana, Oryza, Panieum, Pannesetum, Persea, Pisum, Pyrus, Prunus, Raphanus, Secale, Senecio, Sinapis, Solanum, Sorghum, Trigonella, Triticum, Vitis, Vigna, and, Zea.

### EXAMPLES

The following examples are offered to illustrate, but not to limit the claimed invention.

### Example 1

In this study, viruses and heterologous RNA were used to identify cis-acting elements and proteins necessary and sufficient for directed cell-to-cell transport of RNA. As cell-to-cell and long-distance transport of RNA involves plasmodesmata, these results provide a foundation for dissection of endogenous nucleic acid transport processes. Potexviruses were used to identify cis-acting elements and the specific protein machinery required to mediate the cell-to-cell trafficking of a heterologous RNA molecule.

Potexviruses employ four proteins, the three members of the triple gene block (TGBp1-3) and the coat protein (CP), for cell-to-cell transport of their infectious RNA (Lough, T. J. et al. Mol. Plant-Microbe Interact. 11:801-814 (1998)). Delivery of the movement protein, TGBp1, to the plasmodesmata is mediated by TGBp2 and TGBp3 (Saito, Virology 176:329-336 (1990)). In the absence of either TGBp1 or CP the virus remains restricted to the initially infected cell (Table 1). That both TGBp1 and CP are required for the intercellular movement of infectious RNA was illustrated by co-bombardment experiments in which movement-defective mutants were rescued by a plasmid encoding the corresponding wild-type gene (Table 1). As infection is initiated at the single cell level (by transient nuclear expression of constructs that are fused to the 35S promoter), the extensive cell-to-cell movement of the virus proves that both TGBp1 and CP are co-translocated through plasmodesmata with the infectious RNA.

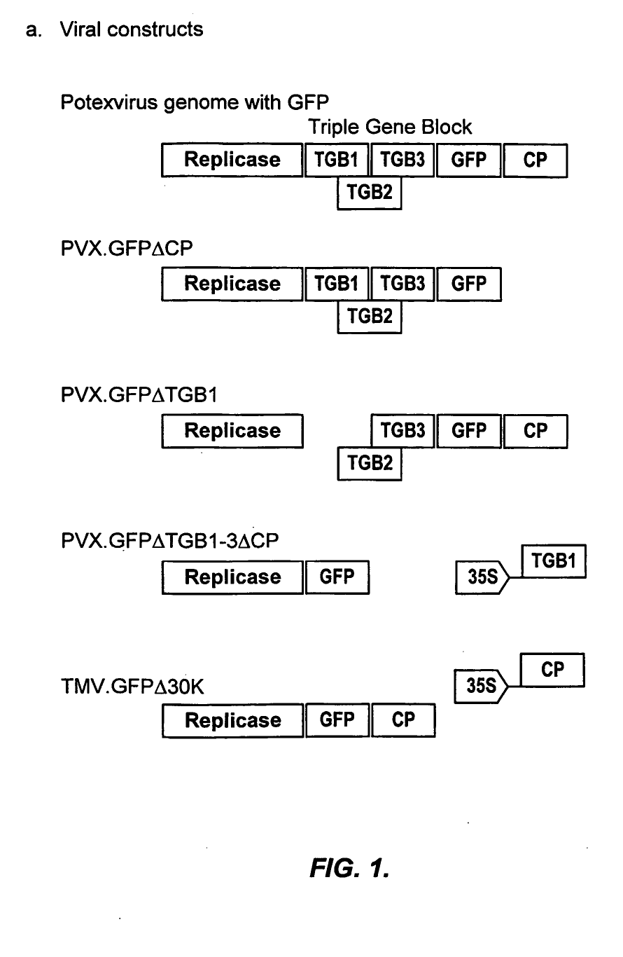

The absolute requirement for specific cognate protein—protein interaction was established by initiating infection with PVX.GFPΔCP, PVX.GFPΔTGB1, or PVX.GFPΔTGB1-3ΔCP (FIG. 1a), on transgenic plants expressing WC1MV CP, TGBp1 or TGBp1-3 plus CP (Table 1). The capacity of WC1MV TGBp1-3 plus CP expressing plants to rescue cell-to-cell movement of WC1MVΔTGB1-3ΔCP, but not the equivalent PVX mutant, demonstrates that the presence of a fully assembled and functional WC1MV movement complex exhibits cognate RNA specificity (Table 1). Equivalent experiments conducted using transgenic plants in which any of these four proteins was missing resulted in restriction of viral infection to the initially bombarded cell (Table 1). Parallel experiments performed with movement-defective TMV (FIG. 1a) further confirmed that the potexvirus movement complex exhibits cognate RNA specificity (Table 1).

To prove that zip codes allow for cell-to-cell transport of RNA, putative cis-acting viral elements were fused to the heterologous GFP reporter. Analysis of GFP fluorescence provided an effective assay for the action of these elements within the viral genome. When such constructs were introduced alone, into plant tissues, fluorescence was confined to the initially bombarded cell. In contrast, co-bombardment with the cognate potexvirus resulted in extensive cell-to-cell movement and expression of the engineered RNA translocator (ERT) (Tables 1 and 2). Confirmation that the RNA was translocated, via plasmodesmata, was provided by the absence of fluorescence in guard cells which lack functional plasmodesmata in mature leaves. As expected, a GFP reporter lacking cis-acting sequences was always confined to the initially bombarded cell (Tables 1 and 2).

Specificity of the cis-acting elements in mediating ERT cell-to-cell movement was further confirmed by co-bombardment experiments using heterologous potexviruses. Only cognate virus-ERT combinations resulted in the development of detectable fluorescent foci (Table 1). In addition, RT-PCR analysis of tissues from such experiments confirmed the hypothesis that cis-acting elements potentiated the cell-to-cell trafficking of an ERT. Again, only in the case where the cognate viral movement machinery and coat protein recognized the ERT were we able to obtain evidence of specific amplification and RNA movement.

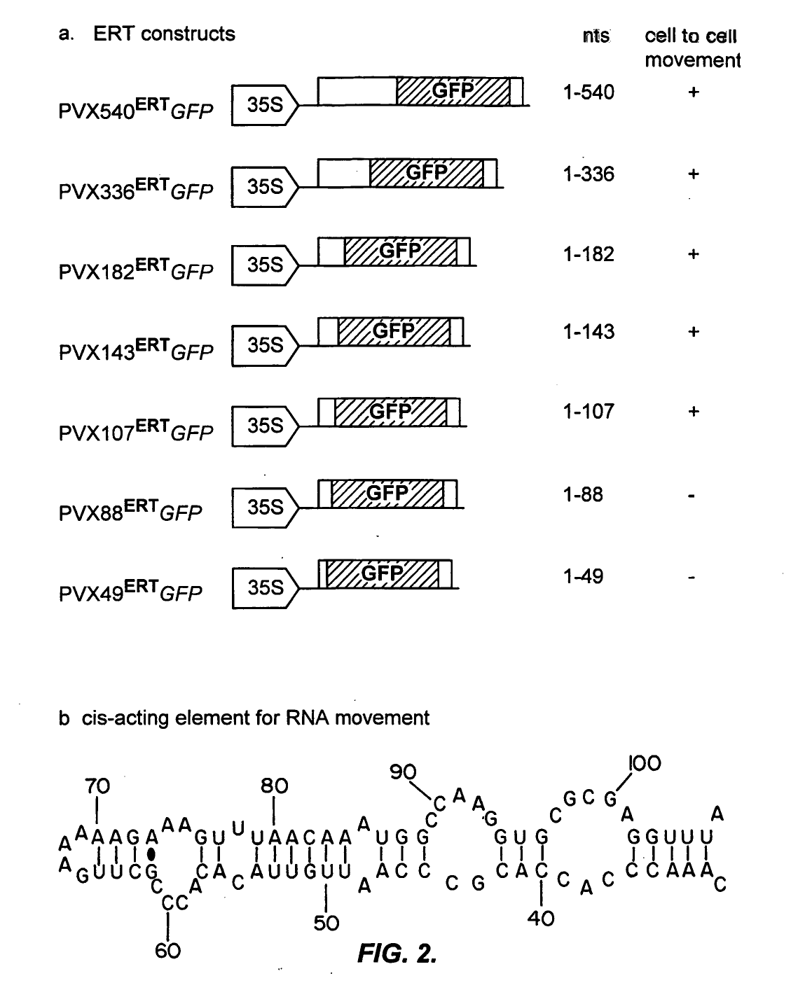

Mapping experiments have localized the cis-acting element within nucleotides 1-107 of the potexvirus genome (FIG. 2a, Table 2). Once again, RT-PCR analysis (using _{PVX107}ERT_{GFP}) established that only cognate virus-ERT combinations resulted in the development of detectable fluorescent foci. Restriction of infection foci of _{PVX182}ERT_{GFP}, _{PVX143}ERT_{GFP} or _{PVX107}ERT_{GFP} co-bombarded with 35S-PVX to the initially bombarded cell indicated either transient expression derived from ERT alone or replication of ERT variants that were incapable of cell-to-cell movement (Table 2). Conserved RNA secondary structures, required for efficient replication, have been defined within nucleotides 1-230 (Miller, J. Mol. Biol. 284:591-608 (1998)). The _{PVX107}ERT_{GFP} construct retained the capacity for cell-to-cell movement. This supported the conclusion that a structural element required for replication (Miller, et al., J. Mol. Biol. 284, 591-608 (1998)), between nucleotides 32-106 (FIG. 2b), is the cis-acting element essential for cognate virus-ERT cell-to-cell RNA movement.

The present work provides irrefutable evidence for the action of specific cis-acting elements, functioning as zip-codes, for selective recognition and transport of RNA between plant cells. These sequences are located in the RNA and their presence in the ERT is necessary and sufficient for recognition and intercellular transport. Without intending to limit the scope of the invention, a model consistent with these findings involves: (a) recognition of the cis-acting element(s) by the CP, (b) subsequent recruitment of TGBp 1, and (c) interaction of this CP-TGBp 1-RNA complex with the endogenous plasmodesmal machinery for cell-to-cell transport of RNA. It remains possible that certain cellular boundaries exhibit differing levels of cognate protein-RNA specificity to permit cell-to-cell translocation of RNA. This may explain why for certain viruses, including tobacco mosaic virus and red clover necrotic mosaic virus, entry into the plant vascular system requires both the movement protein and coat protein.

Methods

Viral Constructs

Constructs based on 35S-PVX.GFP-CP, 35S-TGB1 and 35S-CP were as previously described. See, e.g., Lough, T. J. et al., supra. The infectious constructs for PVX.GFPΔTGB1-3ΔCP and TMV-GFPΔ30K are described in Voinnet, Cell 103:157-167 (2000) and Shivprasad, et al. Virology 255:312-23 (1999), respectively. Clones including WC1MV-GFPΔTGB1, WC1MV-GFPΔCP and WC1MV-GFPΔTGB1-3ΔCP were based on WC1MV-GFP (Lough, T. J. et al., supra). Engineered RNA translocator (ERT) constructs were derived from genomic sequences of either PVX or WC1MV. A GFP reporter (mGFP5 (Shivprasad, et al.)) was inserted inframe into the PVX genome at BamHI-HpaI (548-6065, _{PVX548}ERT_{GFP}). The extreme 370 nucleotides (6065-6435) remained fused to the _{PVX548}ERT_{GFP}. A further series of ERT constructs incorporating 336, 182, 143, 107, 88 or 49 5′ nucleotides (_{PVX336}ERT_{GFP}, _{PVX182}ERT_{GFP}, _{PVX143}ERT_{GFP}, _{PVX107}ERT_{GFP}, _{PVX88}ERT_{GFP} and _{PVX49}ERT_{GFP}, respectively) were made by PCR using _{PVX548}ERT_{GFP} as the PCR template. An equivalent ERT construct was produced using WC1MV sequences with GFP inserted in frame at SacII-NruI (872-5691, _{WC1MV872}ERT_{GFP}). Again, the extreme 155 nucleotides (5692-5846) were fused to the _{WC1MV872}ERT_{GFP}. All ERT constructs were confirmed by sequence analysis. Nucleotide numbering of PVX and WC1MV sequences was according to Haseloff, Proc. Natl. Acad. Sci. USA. 94:2122-2127 (1997) and Huisman, et al. J. Gen. Virol. 69:1789-1798 (1988), respectively. All ERT constructs were fused to the 35S promoter such that the first transcribed base was identical to that of the corresponding virus. The infectious clone 35S-PVX was engineered by deletion of GFP (EagI-BstBI) from 35S-PVX-GFP-CP. The 35S promoter was fused to the WC1MV genome by insertion at the StuI site of pCass2 (Shi, et al., J. Gen. Virol. 78:1181-1185 (1997)).

Microprojectile Bombardment, Micro-Injection and Fluorescence Analysis

Infectious plasmids were introduced into Nicotiana benthamiana epidermal cells of mature leaves as described in Lough, T. J. et al. Transgenic plants expressing WC1MV products are as described in Lough, T. J. et al. Mol. Plant-Microbe Interact. 11:801-814 (1998) and Lough, T. J. et al. Mol. Plant-Microbe Interact. 13:962-974 (2000). Analysis of the spatial distribution of GFP in plant tissues was performed 4-7 days post bombardment using either confocal laser scanning microscopy (Leica model TCS 4D) or epifluorescence microscopy (Leica MPIII stereomicroscope equipped with a DC200 digital camera). Mesophyll cells of mature N. benthamiana leaves were used for micro-injection studies. Tissue preparation, the micro-injection system employed, preparation of fluorescently labeled RNA for pressure-mediated injection and image analysis were carried out as described in Balachandran, Proc. Natl. Acad. Sci. USA 94:14150-14155 (1997) and Kragler, et al. Plant J. 15:367-381 (1998).

RT-PCR Analysis

Poly(A)+ mRNA was extracted from plant tissues using oligo(dT) magnetic beads (Dynal). One step RT-PCR (Gibco-BRL) reactions were performed on 5-fold serial dilutions of RNA. PVXERTGFβ specific reactions were performed in a 20 ul reaction volume using 5 pmol of each primer (5′-ATCTAGCAGGATCCCAGTAAAGGAGAAGAACTTTT-3′ and 5′-TAGGCGTCGGTTATGTAGACGTAGT-3′). RbcS was used as an internal control with reactions as performed above using 5 pmol of each primer (5′-AATTGCTCCTGGCTCAAATC-3′ and 5′-GCTTCCTCAGTTCTTTCCTC-3′). RT-PCR was performed using: 1 cycle of 50° C. for 30 minutes, 94° C. for 2 minutes; 30 cycles of 94° C. for 15 sec, 55° C. for 30 seconds and 68° C. for 1 minute; followed by 1 cycle of 72° C. for 5 minutes.

TABLE 1
Proteins and cis-acting elements required for directed cell-to-cell transport of RNA

| | Transgenically | | | | | | |
|---|---|---|---|---|---|---|---|
| Plasmid/transcripts | expressed proteins | Infection foci^{b} | | | | | |
| used in transient | or co-bombarded | [number of foci, (%)] | | | | | |
| expression assays | plasmids^{a} | 1 cell | | 2-9 cells | | ≧10 cells | |
| p35S-PVX.GFP-CP | — | | | | | 1041 | (100) |
| WCIMV-GFP | — | | | | | 673 | (100) |
| p35S-PVX.GFPΔTGB1 | — | 353 | (100) | | | | |
| p35S-PVX.GFPΔCP | — | 200 | (100) | | | | |
| p35S-PVX.GFPΔTGB1 | - + p35S-PVX TGB1 | 452 | (24) | 979 | (52) | 452 | (24) |
| p35S-PVX.GFPΔCP | - + p35S-PVX CP | 352 | (18) | 919 | (47) | 684 | (35) |
| p35S-PVX.GFPΔCP | CP | 174 | (100) | | | | |
| p35S-PVX.GFPΔTGB1 | TGBp1 | 631 | (100) | | | | |
| p35S-PVX.GFPΔTGB1-3ΔCP | TGBp1-3 & CP | 609 | (100) | | | | |
| WClMV-GFPΔTGB1-3ΔCP | — | 148 | (100) | | | | |
| WClMV-GFPΔTGB1-3ΔCP | TGBp1-3 & CP | 94 | (6) | 552 | (35) | 930 | (59) |
| p35S-PVX.GFPΔTGB1-3ΔCP | TGBp1-3 & CP | 609 | (100) | | | | |
| WCIMV-GFPΔTGB1-3ΔCP | TGBp1-3 | 230 | (100) | | | | |
| WCIMV-GFPΔTGB1-3ΔCP | TGBp2-3 & CP | 122 | (100) | | | | |
| TMV-GFPΔ30K | — | 71 | (100) | | | | |
| TMV-GFPΔ30K | TMV 30K | | | | | 139 | (100) |
| TMV-GFPΔ30K | TGBp1-3 & CP | 215 | (100) | | | | |
| p35S-WCIMV | P35S-_{WCIMV872}ERT_{GFP} | | | | | 458 | (100) |
| p35S-PVX | P35S-_{PVX548}ERT_{GFP} | | | | | 1629 | (100) |
| p35S-WCIMV | P35S-GFP | 5210 | (100) | | | | |
| p35S-PVX | P35S-GFP | 1851 | (100) | | | | |
| — | P35S-GFP | 4924 | (100) | | | | |

^{a}Wildtype WClMV proteins expressed by transgenic Nicotiana benthamiana plants unless otherwise indicated.
^{b}Number and size of individual infection foci (number of cells exhibiting fluorescence) recorded 4-7 days post bombardment.

TABLE 2
Localization of minimal sequence required for directed cell-to-cell
transport of RNA

| Plasmid/transcripts | | Infection foci^{a} | | | |
|---|---|---|---|---|---|
| used in transient | Co-bombarded | [number of foci, (%)] | | | |
| expression assays | plasmid | 1 cell | | 10 cells | |
| — | P35S-_{PVX336}ERT_{GFP} | nd^{b} | | | |
| — | P35S-_{PVX182}ERT_{GFP} | 5 | (100) | | |
| — | P35S-_{PVX143}ERT_{GFP} | 18 | (100) | | |
| — | P35S-_{PVX107}ERT_{GFP} | 6 | (100) | | |
| — | P35S-_{PVX88}ERT_{GFP} | 1 | (100) | | |
| — | P35S-_{PVX49}ERT_{GFP} | 4 | (100) | | |
| p35S-WClMV | P35S-_{PVX336}ERT_{GFP} | nd | | | |
| p35S-WClMV | P35S-_{PVX182}ERT_{GFP} | 5 | (100) | | |
| p35S-WClMV | P35S-_{PVX143}ERT_{GFP} | 82 | (100) | | |
| p35S-WClMV | P35S-_{PVX107}ERT_{GFP} | 443 | (100) | | |
| p35S-WClMV | P35S-_{PVX88}ERT_{GFP} | 664 | (100) | | |
| p35S-WClMV | P35S-_{PVX49}ERT_{GFP} | 580 | (100) | | |
| p35S-PVX | P35S-_{PVX336}ERT_{GFP} | | | 1260 | (100) |
| p35S-PVX | P35S-_{PVX182}ERT_{GFP} | 10 | (2) | 576 | (98) |
| p35S-PVX | P35S-_{PVX143}ERT_{GFP} | 203 | (36) | 359 | (64) |
| p35S-PVX | P35S-_{PVX107}ERT_{GFP} | 250 | (56) | 170 | (38) |
| p35S-PVX | P35S-_{PVX88}ERT_{GFP} | 510 | (100) | | |
| p35S-PVX | P35S-_{PVX49}ERT_{GFP} | 93 | (100) | | |

^{a}Number and size of individual infection foci (number of cells exhibiting fluorescence) recorded 4-7 days post bombardment.
^{b}nd, not detected

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

1. An expression cassette comprising a polynucleotide linked to a heterologous nucleic acid, wherein:
the polynucleotide comprises an ERT sequence at least 70% identical to a sequence selected from the group consisting of SEQ ID NO:1, SEQ ID NO:5, SEQ ID NO:22 and SEQ ID NO:23,
the polynucleotide does not include a nucleotide sequence encoding an active PVX replicase, movement protein or coat protein; and
introduction of the expression cassette into a plant tissue expressing TGBp 1-3 and a PVX coat protein results in transport between cells of an RNA molecule comprising ERT.

2. The expression cassette of claim 1, wherein the polynucleotide comprises an ERT sequence at least 70% identical to SEQ ID NO:1.

3. The expression cassette of claim 1, wherein the polynucleotide comprises an ERT sequence at least 70% identical to SEQ ID NO:5.

4. The expression cassette of claim 3, wherein the ERT sequence is SEQ ID NO:5.

5. The expression cassette of claim 1, wherein the ERT sequence comprises a polynucleotide at least 70% identical to SEQ ID NO:1.

6. The expression cassette of claim 5, wherein the ERT sequence is SEQ ID NO:1.

7. The expression cassette of claim 5, wherein the ERT sequence comprises SEQ ID NO:2.

8. The expression cassette of claim 5, wherein the ERT sequence comprises SEQ ID NO:3.

9. The expression cassette of claim 5, wherein the ERT sequence comprises SEQ ID NO:4.

10. The expression cassette of claim 1, further comprising a promoter operably linked to the polynucleotide.

11. The expression cassette of claim 10, wherein the promoter is constitutive.

12. The expression cassette of claim 10, wherein the promoter is inducible or tissue-specific.

13. A cell comprising:
(a) an RNA molecule comprising a polynucleotide linked to a heterologous nucleic acid, wherein the polynucleotide comprises an ERT sequence at least 70% identical to a sequence selected from the group consisting of SEQ ID NO:1, SEQ ID NO:5, SEQ ID NO:22 and SEQ ID NO:23;
(b) PVX movement proteins TGBp1-3; and
(c) a PVX coat protein.

14. The cell of claim 13, wherein the polynucleotide comprises an ERT sequence at least 70% identical to SEQ ID NO:1.

15. The cell of claim 13, wherein the polynucleotide comprises an ERT sequence at least 70% identical to SEQ ID NO:5.

16. The cell of claim 13, which is a plant cell.

17. The cell of claim 16, wherein the plant cell is part of a plant.

18. The cell of claim 14, wherein the ERT sequence comprises SEQ ID NO:1.

19. The cell of claim 14, wherein the ERT sequence comprises SEQ ID NO:2.

20. The cell of claim 14, wherein the ERT sequence comprises SEQ ID NO:3.

21. The cell of claim 14, wherein the ERT sequence comprises SEQ ID NO:4.

22. A method of mobilizing RNA molecules between cells in a plant, the method comprising,
expressing an RNA molecule in a plant cell, the RNA molecule comprising an ERT sequence at least 70% identical to a sequence selected from the group consisting of SEQ ID NO:1, SEQ ID NO:5, SEQ ID NO:22 and SEQ ID NO:23, wherein the ERT sequence is linked to a heterologous polynucleotide.

23 The method of claim 22, the method further comprising expressing PVX movement proteins TGBp1-3 and a PVX coat protein in the plant cell.

24. The method of claim 22, wherein the plant cell expressing the RNA molecule is contained in a plant tissue that is grafted onto the plant.

25. The method of claim 24, wherein the plant is not transgenic.

26. The method of claim 22, wherein the PVX movement and coat proteins are expressed from a viral vector.

27. The method of claim 22, wherein the PVX movement and coat proteins are encoded by a polynucleotide integrated into the plant genome.

28. The method of claim 22, wherein the ERT sequence comprises a polynucleotide at least 70% identical to SEQ ID NO:1.

29. The method of claim 22, wherein the ERT sequence comprises a polynucleotide at least 70% identical to SEQ ID NO:5.

30. The method of claim 28, wherein the ERT sequence comprises SEQ ID NO:1.

31. The method of claim 29, wherein the ERT sequence comprises SEQ ID NO:5.

32. The method of claim 28, wherein the ERT sequence comprises SEQ ID NO:2.

33. The method of claim 28, wherein the ERT sequence comprises SEQ ID NO:3.

34. The method of claim 28, wherein the ERT sequence comprises SEQ ID NO:4.

35. The method of claim 22, wherein the TGBp1-3 and the PVX coat protein are expressed from a viral genome.

36. The method of claim 22, wherein the TGBp1-3 and the PVX coat protein are expressed from an integrated transgene.

37. A method of identifying a nucleic acid sequence that is transported between cells in a plant, the method comprising,
providing at least one polynucleotide comprising a nucleic acid sequence linked to a reporter gene;
introducing the polynucleotide into a target plant cell in the plant; and
determining whether the reporter gene is expressed in plant cells in the plant other than the target plant cell, thereby identifying a nucleic acid sequence that is transported between cells in a plant.

38. The method of claim 37, wherein the reporter gene is selected from green fluorescence protein, luciferase and P-glucuronidase.

39. The method of claim 37, wherein the nucleic acid sequence is from a plant.

40. A method of mobilizing RNA molecules between cells in a plant, the method comprising,
expressing an RNA molecule in a plant cell, the RNA molecule comprising the nucleic acid sequence identified in claim 37 linked to a heterologous polynucleotide.
